(19) United States
(12) Patent Application Publication
(10) Pub. No.: US 2024/0167966 A1
(43) Pub. Date: May 23, 2024

(54) A METHOD FOR MAPPING AN INTERNAL STRUCTURE OF A SAMPLE

(71) Applicant: THE AUSTRALIAN NATIONAL UNIVERSITY, Australian Capital Territory (AU)

(72) Inventors: Roland FLEDDERMANN, Australian Capital Territory (AU); Geoff CAMPBELL, Australian Capital Territory (AU); Glenn MYERS, Australian Capital Territory (AU); Shane LATHAM, Australian Capital Territory (AU); Zixin LIANG, Australian Capital Territory (AU); Jong CHOW, Australian Capital Territory (AU); Adrian SHEPPARD, Australian Capital Territory (AU)

(73) Assignee: THE AUSTRALIAN NATIONAL UNIVERSITY, Australian Capital Territory (AU)

(21) Appl. No.: 18/277,221

(22) PCT Filed: Feb. 15, 2022
(86) PCT No.: PCT/AU2022/050102

§ 371 Date: Aug. 14, 2023

(30) Foreign Application Priority Data
Feb. 15, 2021 (AU) 2021900371

(51) Int. Cl. G01N 21/95; G01N 21/47; G01N 21/64; G01N 21/88; G01N 33/38; G16C 20/20; G16C 60/00

(52) U.S. Cl. CPC G01N 21/95; G01N 21/4795; G01N 21/6456; G01N 21/8806; G01N 33/381; G16C 20/20; G16C 60/00; G01N 2021/646

(57) ABSTRACT

The disclosure provides a method (200) for determining one or more features associated with an internal structure of a gemstone, the gemstone being at least partially transmissive to electromagnetic radiation. The method comprises directing electro-magnetic radiation towards the gemstone using a source of incident electromagnetic radiation; in response to directing electromagnetic radiation, detecting electromagnetic radiation using an optical detecting means, including detecting electromagnetic radiation following an interaction between the gemstone and the incident electromagnetic radiation; and processing the detected electromagnetic radiation, wherein the processing accounts for a determination of an external surface geometry of the gemstone and for refraction and reflection effects due to the external surface geometry of the gemstone, and obtains information indicative of the one or more features associated with the internal structure of the gemstone. A system for determining one or more features associated with an internal structure of a gemstone is also provided.

### TECHNICAL FIELD

The present invention relates to a method for mapping an internal structure of a sample. More particularly, although not exclusively, the present invention relates to a method for obtaining a three-dimensional map of an internal structure of a material having a high refractive index such as a gemstone.

### BACKGROUND

Optical projection tomography (OPT) is a method to obtain a volumetric image of an optically transmissive sample material by recording two-dimensional images (projections) of light that has been transmitted through the object from multiple angles. Electromagnetic radiation in a range of optical wavelengths between approximately 400 and 1600 nm may be used to produce the light projections. Regions of a sample material that absorb or scatter light will cast shadows on the imaging detector. The three-dimensional structure of the sample material is then computationally reconstructed from the shape and relative darkness of the shadows cast, as the sample material is viewed from many different angles.

Some related optical tomography techniques, such as for example optical emission tomography also known as optical emission computed tomography, may use images formed by light that is emitted or scattered from a side- or front-illuminated object, instead of shadows cast by transmitted light, to reconstruct a three-dimensional volume of an object using a similar computational approach.

Operating at optical wavelengths in the visible and infrared range however has the detrimental effect of introducing complexity in the interaction between the sample material and optical light. In standard OPT applications including imaging of a gemstone, such as diamond having a rough external surface, the gemstone is submerged in a refractive index matching fluid or embedded in a refractive index matching solid to reduce scattering and make light paths entering the gemstone approximately straight lines, i.e. reduce reflection and refraction effects to a level where they become negligible for the reconstruction process. This submersion of the gemstone or other transparent material in a refractive index matching fluid or solid adds complexity to the process of obtaining a volumetric image of the material and, in the case of materials having high refractive indices (such as gemstones) may require toxic fluids or solids.

Due to the high refractive index and dispersion within the gemstone, it is not possible to use a single refractive index matching fluid or solid that has the same refractive index at different wavelengths, such as all wavelengths in the visible range. As a result, to achieve refractive index matching at multiple different wavelengths, multiple submersion materials (fluid or solid) need to be used, however it may not be possible to achieve sufficiently accurate index matching at all wavelengths. A determination of chromatic properties of a gemstone or other material, resulting in, for example, a bulk colour of a gemstone or other material, and/or a colour of an inclusion/defect within the gemstone or other material, demands probing the sample with different wavelengths of the incident light. Thus, in light of the experimental challenge faced when performing measurements at different wavelengths, available techniques present a limitation for a determination of chromatic properties in a gemstone.

### Summary

In accordance with a first aspect of the present invention, there is provided a method for determining one or more features associated with an internal structure of a gemstone, the gemstone being at least partially transmissive to electromagnetic radiation, the method comprising:
directing electromagnetic radiation towards the gemstone using a source of incident electromagnetic radiation;
in response to directing electromagnetic radiation, detecting electromagnetic radiation using an optical detecting means, including detecting electromagnetic radiation following an interaction between the gemstone and the incident electromagnetic radiation; and
processing the detected electromagnetic radiation, wherein the processing accounts for a determination of an external surface geometry of the gemstone and for refraction and reflection effects due to the external surface geometry of the gemstone, and obtains information indicative of the one or more features associated with the internal structure of the gemstone.

In one embodiment, the method further comprises determining the external surface geometry of the gemstone. In one embodiment, processing the detected electromagnetic radiation comprises determining the external surface geometry of the gemstone using the detected electromagnetic radiation.

In one embodiment, directing electromagnetic radiation towards the gemstone comprises directing electromagnetic radiation towards the gemstone from a plurality of different incident directions relative to the external surface geometry of the gemstone.

In this embodiment, detecting electromagnetic radiation may comprise detecting electromagnetic radiation for each incident direction.

In one embodiment, processing the detected electromagnetic radiation comprises generating an output associated with a three-dimensional distribution of an optical property within the gemstone, the three-dimensional distribution of the optical property being indicative of a three-dimensional distribution of the one or more features associated with the internal structure of the gemstone.

Generating the output may comprise applying an iterative algorithm.

In one embodiment, the method further comprises using the output to generate a three-dimensional graphical representation of the three-dimensional distribution of the one or more features associated with the internal structure of the gemstone.

In one embodiment, processing the detected electromagnetic radiation comprises generating, based on the determined external surface geometry, a model of a simulated propagation of simulated electromagnetic radiation between the source of incident electromagnetic radiation and the optical detecting means and via a simulated homogeneous sample, wherein interaction between the simulated electromagnetic radiation and the simulated homogeneous sample is accounted for, the simulated homogenous sample comprising a simulated external surface having the determined external surface geometry of the gemstone and wherein the simulated homogeneous sample has a homogeneous refractive index. In the embodiment for which the method comprises directing electromagnetic radiation towards the gemstone from a plurality of different incident directions relative to the external surface geometry of the gemstone, the model of simulated propagation of the simulated electromagnetic radiation may be generated for each incident direction.

Further, the method may comprise modelling simulated refraction and attenuation of the simulated electromagnetic radiation at a plurality of virtual surface boundaries of the simulated homogenous sample. The method may further comprise modelling simulated reflection of the simulated electromagnetic radiation at a plurality of virtual surface boundaries of the simulated homogenous sample. The method may further comprise modelling, based on the model of the simulated propagation, a simulated polarisation state of the simulated electromagnetic radiation propagating between the source of incident electromagnetic radiation and the optical detecting means and via the simulated homogeneous sample, wherein modelling the simulated polarisation state accounts for an interaction between the simulated electromagnetic radiation and respective virtual surface boundaries at the external surface of the simulated homogeneous sample.

The method may further comprise modelling a shape and an intensity of a simulated beam of incident electromagnetic radiation. In this embodiment, the method may comprise using the modelled shape and intensity of the simulated beam of incident electromagnetic radiation to determine a size of a region of interaction of the simulated beam of incident electromagnetic radiation with a virtual external surface boundary of the simulated homogeneous sample.

In one embodiment, processing the detected electromagnetic radiation comprises using a computed tomography process.

The one or more features may include at least one or more of the following: a defect; an inclusion; an impurity; a chromatic property; a polarisation property.

In one embodiment, the method comprises directing electromagnetic radiation towards the gemstone at a minimum of two different wavelengths.

Detecting electromagnetic radiation may comprise detecting electromagnetic radiation for each wavelength. Processing the detected electromagnetic radiation may comprise processing the detected electromagnetic radiation for at least two wavelengths, wherein information indicative of one or more chromatic properties associated with the internal structure of the gemstone can be obtained.

In one embodiment, the source of incident electromagnetic radiation comprises a diffuse source of electromagnetic radiation.

In one embodiment, the method further comprises moving the gemstone, the source of incident electromagnetic radiation and the optical detecting means relative to each other.

In one embodiment, detecting electromagnetic radiation comprises detecting electromagnetic radiation transmitted from the gemstone.

In another embodiment, detecting electromagnetic radiation comprises detecting electromagnetic radiation scattered and/or reflected and/or caused by fluorescence from within the gemstone.

In accordance with a second aspect of the present invention, there is provided a system for determining one or more features associated with an internal structure of a gemstone, the gemstone being at least partially transmissive to electromagnetic radiation, the system comprising:
a source of incident electromagnetic radiation configured to emit electromagnetic radiation towards the gemstone;
an optical detecting means configured to detect electromagnetic radiation including electromagnetic radiation detected following an interaction between the gemstone and the incident electromagnetic radiation; and
a processor configured to:
receive a first input associated with the detected electromagnetic radiation;
receive a second input associated with an external surface geometry of the gemstone; and
generate an output indicative of a three-dimensional distribution of an optical property within the internal structure of the gemstone, the optical property being associated with an interaction between the gemstone and the incident electromagnetic radiation, the three-dimensional distribution of the optical property being indicative of a three-dimensional distribution of the one or more features within the internal structure of the gemstone;
wherein the output is generated based on the first input, the second input, and accounting for refraction and reflection effects due to the external surface geometry of the gemstone.

In one embodiment, the system is configured such that the source of electromagnetic radiation emits electromagnetic radiation towards the gemstone from a plurality of different incident directions relative to the external surface geometry of the gemstone.

In one embodiment, the processor is further configured to generate, using the output, a three-dimensional graphical representation of the one or more features associated with the internal structure of the gemstone.

In one embodiment, the processor is further configured to:
generate a first model of a simulated homogenous sample, the simulated homogenous sample comprising a simulated external surface having the determined external surface geometry of the gemstone, wherein the simulated homogeneous sample has a homogeneous refractive index;
generate a second model of a propagation of simulated electromagnetic radiation between the source of incident electromagnetic radiation and the optical detecting means and via a simulated homogeneous sample, wherein interaction between the simulated electromagnetic radiation and the simulated homogeneous sample is accounted for; and
use the first model and the second model to generate the output.

In accordance with a third aspect of the present invention, there is provided a computer program comprising executable code configured to cause the process of the system of the second aspect to execute the steps of:
receiving the first input;
receiving the second input; and
generate the output wherein the output is generated based on the first input, the second input, and accounting for refraction and reflection effects due to the external surface geometry of the gemstone.

In accordance with a fourth aspect of the present invention, there is provided a method for determining one or more features associated with an internal structure of a test sample, the method comprising:
directing electromagnetic radiation towards the test sample using a source of incident electromagnetic radiation;
in response to directing electromagnetic radiation, detecting electromagnetic radiation using an optical detecting means, including detecting electromagnetic radiation following an interaction between the test sample and the incident electromagnetic radiation; and
processing the detected electromagnetic radiation, wherein the processing:
   accounts for a determination of an external surface geometry of the test sample and for refraction and reflection effects due to the external surface geometry of the test sample; and
   obtains information indicative of the one or more features associated with the internal structure of the test sample.

In accordance with a fifth aspect of the present invention, there is provided a system for determining one or more features associated with an internal structure of a test sample, the system comprising:
a source of incident electromagnetic radiation configured to emit electromagnetic radiation towards the test sample;
an optical detecting means configured to detect electromagnetic radiation including electromagnetic radiation following an interaction between the test sample and the incident electromagnetic radiation; and
a processor configured to:
receive a first input associated with the detected electromagnetic radiation;
receive a second input associated with an external surface geometry of the test sample; and
generate an output indicative of a three-dimensional distribution of an optical property within the internal structure of the test sample, the optical property being associated with an interaction between the test sample and the incident electromagnetic radiation, the three-dimensional distribution of the optical property being indicative of a three-dimensional distribution of the one or more features within the internal structure of the test sample;
wherein the output is generated based on the first input, the second input, and accounting for refraction and reflection effects due to the external surface geometry of the test sample.

In one embodiment, the test sample comprises a material having a high refractive index.

### BRIEF DESCRIPTION OF THE DRAWINGS

Notwithstanding any other forms which may fall within the scope of the disclosure as set forth in the Summary, specific embodiments will now be described, by way of example only, with reference to the accompanying drawings in which:

FIG. 1(a) shows a simplified schematic representation of a propagation of ray paths of electromagnetic radiation between a source of incident electromagnetic radiation and an optical detecting means wherein the incident electromagnetic radiation is scanned across a gemstone immersed in a refractive index-matching material;

FIG. 1(b) shows a simplified schematic representation of a propagation of ray paths of electromagnetic radiation between a source of incident electromagnetic radiation and an optical detecting means wherein the incident electromagnetic radiation is scanned across a gemstone in the absence of a refractive index-matching material;

FIG. 2 shows a flow chart of a method in accordance with an embodiment of the present invention;

FIG. 3a shows a schematic representation of an optical tomography system in accordance with an embodiment of the present invention;

FIG. 3b shows a schematic representation of an optical tomography system in accordance with another embodiment of the present invention;

FIG. 4 shows a flow chart of an implementation of the method of FIG. 2 in accordance with an embodiment of the present invention;

FIG. 5 shows a schematic representation of a data acquisition process in accordance with an embodiment of the present invention;

FIG. 6 shows a schematic representation of a simulated modelled propagation of simulated electromagnetic radiation in accordance with an embodiment of the present invention;

FIG. 7 shows another schematic representation of a simulated modelled propagation of simulated electromagnetic radiation; and

FIG. 8 shows a schematic representation of a scanning optical system used in accordance with an embodiment of the present invention.

### DETAILED DESCRIPTION

Where reference is made in any one or more of the accompanying drawings to steps and/or features that have the same reference numerals, those steps and/or features have for the purposes of this description the same function(s) or operation(s), unless the contrary intention appears.

In the context of this application, “defect” is intended to mean a naturally or manmade irregularity. A defect may include but is not exclusive to: voids, cracks, mineral inclusions, natural formations, growth patterns, etc.

Embodiments of the present invention aim at providing a method and a system for a refraction-corrected reconstruction of a distribution of one or more features associated with an internal structure of a sample. The sample may be referred to as a test sample, the terms “test sample” being used in the context of this application to refer to a sample that is being under investigation to determine one or more features, such as, however not limited to, inclusions or other defects, associated with its internal structure.

In one specific embodiment, the test sample is a gemstone, and may be a whole gemstone or a cut gemstone. The gemstone may have a complex irregularly shaped external surface geometry and may for example be a non-polished gemstone, such as a non-polished diamond or the like. However, it will be understood that embodiments of the present invention are not limited to the test sample being a gemstone and is also not limited to a sample having a complex irregularly shaped external surface geometry. The test sample may be any object or sample of a transparent or semi-transparent material. The test sample may be any sample comprising a material that is at least partially transmissive to electromagnetic radiation, and may for example, however not limited to, be a sample comprising a crystalline material having a high average refractive index, i.e. an average refractive index above 1.50.

In the context of this application, the “internal structure of the test sample” or “internal structure of the gemstone” is intended to refer to the inside or interior of the test sample or gemstone, i.e. the part of the test sample or gemstone within its envelope beneath the outer surface of the test sample or gemstone. The terms “feature(s) associated with the internal structure of the test sample” are intended to refer to any feature contained within or forming part of the test sample (e.g., gemstone) within the envelope of the test sample (e.g., gemstone) that is indicative of some inhomogeneity inside the test sample. Feature(s) associated with the internal structure or interior of the test sample or gemstone are intended to include structural features contained within or forming part of the test sample within the envelope of the test sample, such as defects, flaws, or inclusions contained within or forming part of the test sample within the envelope of the test sample, as well as other features contained within or forming part of the test sample within the envelope of the test sample such as chromatic features/properties, and polarisation features/properties. The features may be localised elements and/or may be broadly distributed through the interior of the test sample. Further, the features may comprise features with sharp or abrupt boundaries, and/or may comprise a continuous variation of features/properties.

The features may include those that are visible to the naked eye, as well as those that are only visible under levels of magnification.

The terms “inclusion”, “flaw” and “defect” are used in the following interchangeably to indicate an individual discernible irregularity inside the test sample.

Further, in the context of this application, a chromatic property is meant to relate to a wavelength dependence of optical properties of the internal structure of the test sample and is meant to include, however is not limited to, features such as: a colour of an inclusion or other defect; an average colour within the internal structure of the test sample; a continuous variation in colour in the internal structure of the test sample; an abrupt variation in colour in the internal structure of the test sample; a colour or average colour of an inclusion or other defect in the internal structure of the test sample; a colour and/or intensity of light reflected or scattered from, or emitted by, an inclusion or other defect; an average colour and/or intensity associated with absorption or fluorescence from the internal structure of the test sample; a fluorescence of an inclusion or other defect; and/or an average fluorescence associated with the internal structure of the test sample.

In the context of this application, “a polarisation property” is intended to refer to an optical property of the test sample (such as for example however not limited to, a partially transparent material) that depends on, is associated with, or relates to, a polarisation state of a beam of electromagnetic radiation interacting with the test sample. For example (however not limited to), a polarisation property of the test sample may in the context of this application refer to a birefringence of the test sample; a variation in birefringence due to the internal structure of the test sample; a polarisation dependence in scattering or fluorescence from a defect, inclusion, or impurity in the test sample.

The expressions “non-refractive internal structure”, “homogeneous refractive index”, and “homogenous internal structure” are used in the context of this application to indicate an internal structure of a sample that comprises substantially no impurities or flaws that would cause a further refraction of the electromagnetic radiation after it enters the sample, besides a first refraction that occurs at the physical boundary between the air and the sample at the sample surface or envelope. It is noted that a “homogenous internal structure” may cause attenuation, scattering, reflection and/or other optical effects.

Further, the expression “homogeneous sample” is used in the context of this application to mean a sample having a homogenous internal structure.

Embodiments of the present invention generally combine an optical tomography technique with a detailed modelling of reflection and refraction of light rays at the surface of a potentially complex-shaped test sample to obtain information associated with an internal structure of the sample without the need for immersing the test sample in a refractive index matching fluid or solid. More specifically, embodiments of the present invention aim at providing a refraction/reflection-corrected method and a refraction/reflection-corrected system for obtaining information regarding features internal to the test sample, i.e. features associated with an internal structure of a test sample, including three-dimensional (3D) structural features such as inclusions or other defects as well as other features, such as chromatic properties and polarisation properties. The information obtained enabling characterisation of the sample. The information obtained can also enable accurate modelling of the sample. Such modelling can be advantageous to analyse the sample, for example for a purpose such as to assess quality, determine suitability of a gem stone for an intended purpose, and/or options for cutting the stone.

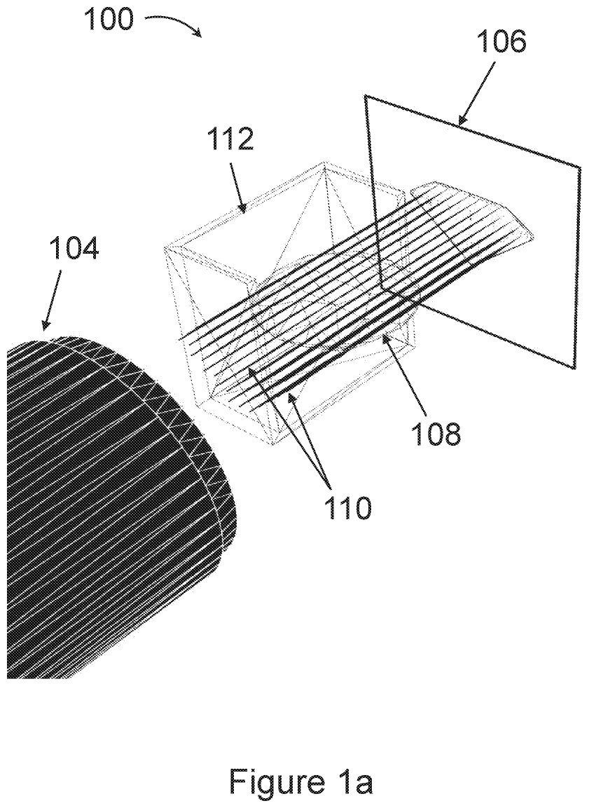

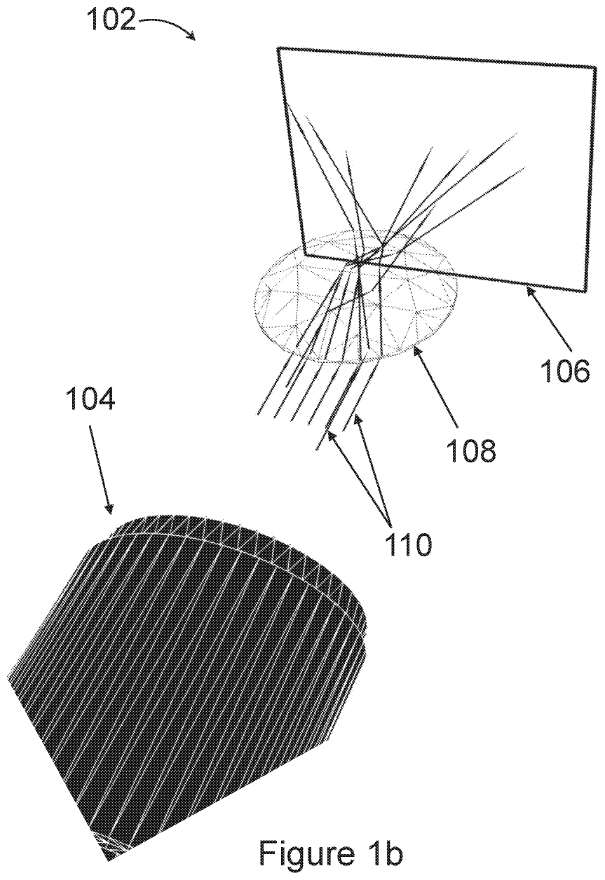

FIGS. 1a and 1b show simplified schematic representations 100 and 102 of ray paths travelling between a source of incident electromagnetic radiation 104 and an optical detecting means 106, wherein the incident electromagnetic radiation is scanned across a gemstone 108 (such as a diamond) in the form of a set of parallel paths 110 for a case with immersion in a refractive index-matching material 112 (FIG. 1a) and a case without immersion in a refractive index-matching material (FIG. 1b). In the case with immersion in a refractive index-matching material, there is minimal/limited refraction or reflection/attenuation at the gemstone surface such that reflection and refraction effects at the gemstone surface can be neglected and known optical tomography methods may be used to build a 3D model of the gemstone interior indicative of the one or more features associated with the internal structure of the gemstone.

In contrast, in the case without immersion in a refractive index matching material, rays of electromagnetic radiation travelling between the source of incident electromagnetic radiation 104 and the optical detecting means 106 may be reflected and/or refracted at the external surface of the gemstone 108. These reflections and refractions at the surface of the gemstone 108 may alter one or more of the direction and intensity of the incident electromagnetic radiation on the features within the gemstone, thereby, influencing the detected electromagnetic radiation. The detected electromagnetic radiation alone cannot provide an accurate characterisation of the gemstone interior due to the reflections and refractions at the surface of the gemstone. However, these reflections and refractions can be accounted for with the detected electromagnetic radiation for building a 3D model of the gemstone interior that is reflection-corrected and refraction-corrected.

It is noted that the sets of parallel incident rays of electromagnetic radiation 110 shown in FIGS. 1a and 1b represent a very small fraction only of a full set of viewing angles that would be required to obtain information indicative of the one or more features associated with the internal structure of the gemstone and build a 3D model of the gemstone interior. It is also noted that although a polished (cut) gemstone is represented, it is only an example simplification and it will be understood that embodiments of the present invention may be applied to any type of test samples, including test samples such as, for example, gemstones having a rough heterogenous non-polished external surface.

The following description will be provided in relation to the test sample being a gemstone. It will however be understood that the following description may be applied to any other type of test sample including any object or sample of a transparent or semi-transparent material, and including any sample comprising a material that is at least partially transmissive to electromagnetic radiation.

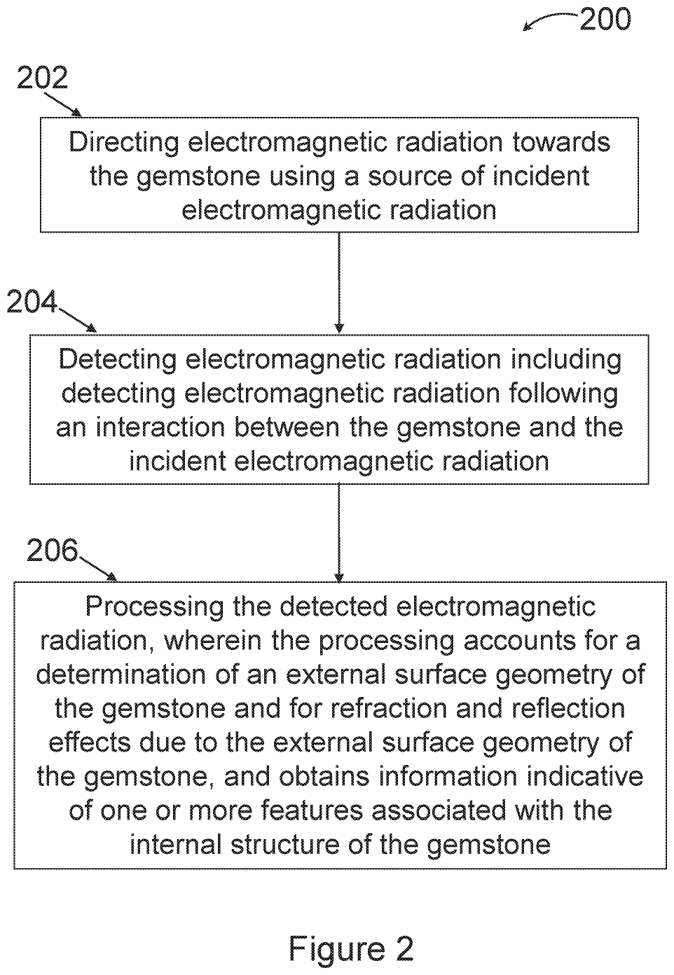

FIG. 2 is a flow chart of a method 200 for determining one or more features associated with an internal structure of a gemstone provided in accordance with an embodiment of the present invention. The method 200 is suitable for identifying one or more features in the internal structure of the gemstone, such as one or more inclusions or other defects, as well as other features such as polarisation features/properties and chromatic features/properties including, however not limited to, one or more of the following: a colour of an inclusion or other defect; an average colour within the internal structure of the gemstone; an abrupt variation or a continuous variation in colour in the internal structure of the gemstone; a colour or average colour of an inclusion or other defect in the internal structure of the gemstone; a colour and/or intensity of light reflected or scattered from, or emitted by, an inclusion or other defect; an average colour and/or intensity associated with absorption or fluorescence from the internal structure of the gemstone; a fluorescence of an inclusion or other defect; and an average fluorescence associated with the internal structure of the gemstone.

At step 202, electromagnetic radiation is directed towards the gemstone using a source of incident electromagnetic radiation.

At step 204, in response to directing the electromagnetic radiation, electromagnetic radiation is detected, using an optical detecting means such as an optical detector or an electro-optical sensor, wherein the detected electromagnetic radiation includes electromagnetic radiation detected following an interaction between the gemstone and the incident electromagnetic radiation.

At step 206, the detected electromagnetic radiation is processed, wherein the processing (i) accounts for a determination of an external surface geometry of the gemstone and for refraction and reflection effects due to the external surface geometry of the gemstone, and (ii) obtains information indicative of the one or more features associated with the internal structure of the gemstone.

The steps of method 200 may be performed simultaneously or may be performed separately.

In one embodiment, the detected electromagnetic radiation is associated with electromagnetic radiation data characteristic of the detected electromagnetic radiation, typically the intensity of the detected electromagnetic radiation, for a given orientation and position of the gemstone.

The terms “electromagnetic radiation” will now be referred to as “EM radiation”.

The data acquisition involves directing EM radiation towards the gemstone from a plurality of different incident directions.

Different optical systems may be used for the acquisition of information (e.g., signals, data, images):

### Scanning Approach

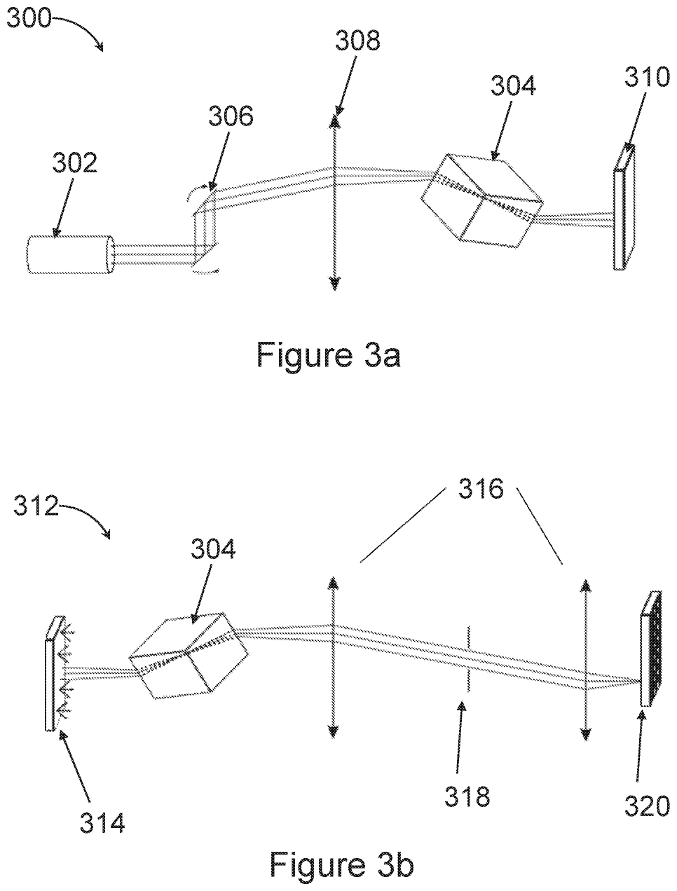

In one embodiment with reference to FIG. 3a, a scanning optical system 300 is used. A laser source 302 is used for directing incident EM radiation towards a gemstone 304. The laser source 302 is adapted for emitting electromagnetic radiation having a known beam shape and may comprise one or more lasers. The laser source 302 is in some embodiments adapted to emit EM radiation at a wavelength (a single wavelength or with a relatively narrow bandwidth) or at a range of wavelengths (if multiple lasers are used or if a tunable laser is used) in the visible wavelength range, for example, between 400 nm and 700 nm. In other embodiments, the laser source 302 may be adapted to emit EM radiation at a wavelength (a single wavelength or with a relatively narrow bandwidth) or at a range of wavelengths (if multiple lasers are used) in the infrared wavelength range or in the ultraviolet wavelength range. It is also envisaged to use any combinations of visible, infrared and/or ultraviolet wavelengths. The scanning optical system 300 further comprises scanning mirrors 306, such as galvanometer scanning mirrors or polygon scanners, which are movable and, in conjunction with a lens system 308, configured to scan the laser beam across at least a portion of the test sample 304. An optical detecting means, e.g., a large area photo-detector 310, such as, for example however not limited to, a 10 mm×10 mm photodiode, is configured and positioned to detect EM radiation received as a result of the emitted EM radiation, the detected EM radiation including EM radiation detected following an interaction between the gemstone 304 and the incident EM radiation. It will however be understood that other alternative or additional suitable and equivalent optical devices, may be used for the scanning system 300, for example Polygon scanners, DLP mirrors/MEMS mirrors, risley prism pairs, deformable mirrors or spatial light modulators.

### Imaging approach

In another embodiment with reference to FIG. 3b, an imaging system 312 is used. A uniform diffuse source 314 of EM radiation is used as source of incident EM radiation directed towards the gemstone 304. For example, a source of EM radiation for which the EM radiation is substantially uniformly diffused (i.e. which emits EM radiation of equal intensity at each source location and into all emission directions (or as evenly a distribution as possible), e.g. a Lambertian emitter) over a large area, for example an area greater than that of a projection of the gemstone. In one specific embodiment, the diffuse source 314 is adapted for emitting EM radiation at one or more wavelengths in the visible wavelength range, e.g. between 400 nm and 700 nm. For example, red, green and blue wavelengths may be used to match colour receptors in a human eye, which may be advantageous for a determination of chromatic properties associated with the internal structure of the gemstone such as colour related properties. The gemstone 304 is directly imaged using a telecentric lens system 316, an aperture 318 positioned at a shared back-focal length of the lenses 316, and an image sensor 320. It will however be understood that other alternative or additional suitable and equivalent optical devices may be used for the imaging system 312, such as, for example, other apertured lens-based systems.

It will further be understood that embodiments of the present invention are not limited to the use of wavelengths in the visible wavelength range and that other wavelengths such as wavelengths in the infrared wavelength range or the ultraviolet wavelength range may be envisaged.

In accordance with embodiments of the present invention, the source of incident EM radiation and the optical detecting means will in the following be referred to as being parts of an optical system.

For acquisition of information (e.g., signals, data, images) for a plurality of directions relative to the gemstone, a motion control of the gemstone may in one embodiment be provided as part of the optical system arrangement, wherein the gemstone is moved relative to the source of incident EM radiation and the optical detecting means (i.e. detector or sensor).

For example, the gemstone may be positioned on a rotation stage, or on two orthogonal rotation stages, to change the relative alignment of the optical system (source of EM radiation and optical detecting means) and gemstone to acquire views from different directions. In particular, by positioning the gemstone on two orthogonal rotation stages, the gemstone is arranged to rotate through two orthogonal rotation axes relative to the source and detector/sensor, whereby the location of the source and detector/sensor relative to the gemstone changes as the gemstone is rotated. The various locations of the source and detector/sensor as a result correspond to points on an imaginary sphere around the gemstone. Such arrangement allows scanning EM radiation through the gemstone for a relatively large number of directions, wherein a large set of data can be achieved to contribute to an accurate characterisation of the one or more features within the internal structure of the gemstone. Alternatively, the source of EM radiation and/or the detector/sensor may be arranged to controllably move relative to the gemstone, or a plurality of sources of EM radiation and/or of optical detecting means (detectors/sensors) may be used to direct EM radiation towards the gemstone from a plurality of different directions and detect EM radiation in response to directing the EM radiation. In another alternative embodiment, one or more stationary sources of EM radiation may be provided but there being an optical device which changes the ray path from the stationary source/s towards the gemstone.

In one embodiment, processing the detected EM radiation comprises using a computed tomography process.

An optical projection tomography system may be used wherein the detected EM radiation is EM radiation that transmitted from the gemstone. With reference to FIGS. 3a and 3b, in response to directing the incident EM radiation, EM radiation is detected (i.e. collected, detected, or sensed) using an optical detecting means such as the large area photo-detector 310 in the scanning approach or the image sensor 320 in the imaging approach. The detected EM radiation includes EM radiation transmitted from the gemstone 304 following an interaction of the incident EM radiation and the gemstone 304.

Alternatively, an optical tomography system may be used wherein the optical detecting means is arranged to detect EM radiation scattered and/or reflected, and/or caused by fluorescence from within the gemstone. In this embodiment, the detected EM radiation does not include transmitted EM radiation but includes any one of scattered EM radiation, reflected EM radiation or fluorescence. The source 302, 314 may not be in line with the optical detecting means 310, 320, and may for example be placed off to the side relative to the optical detecting means or may be placed off in any transversal position relative to the optical detecting means.

The different arrangements according to embodiments of the present invention may be used for the determination of the one or more features (including inclusions and other defects, chromatic properties, etc.) associated with an internal structure of a gemstone or test sample, and the respective acquired information (e.g., signals, data, images) using the different arrangements may be combined into a merged dataset and/or used in a complementary manner.

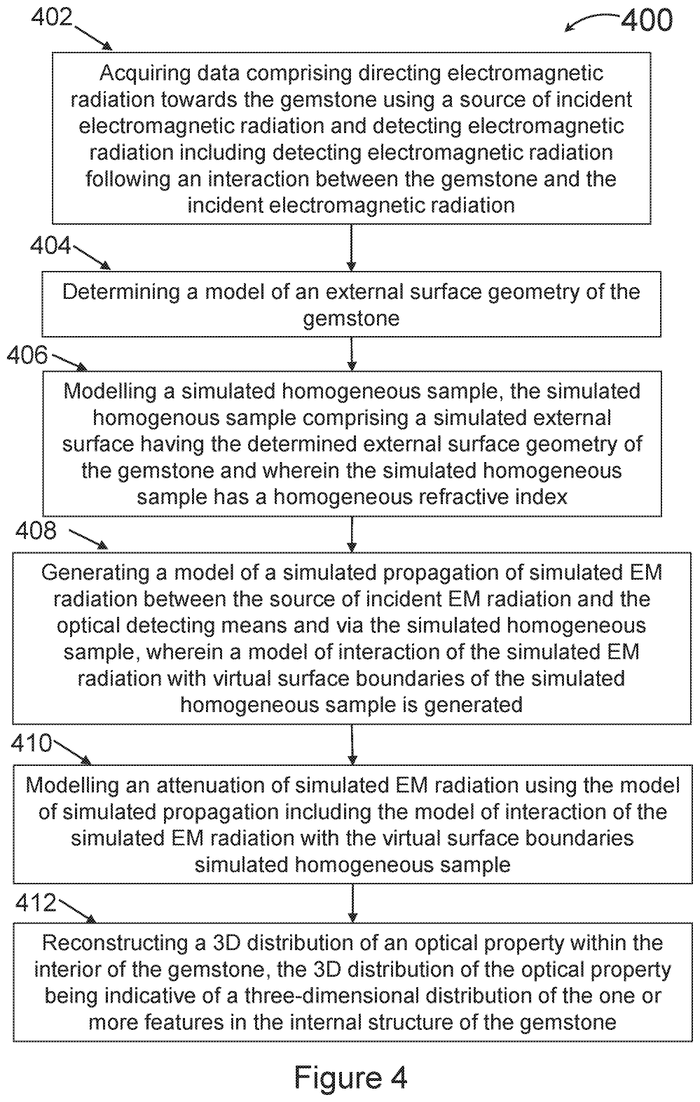

With further references to FIGS. 4 to 8, the steps of the method 200 will now be described for a specific embodiment wherein the test sample is a gemstone and wherein the acquisition of information (e.g., signals, data, images) indicative of the one or more features (including defects, inclusions, impurities, chromatic properties, polarisation properties) associated with the internal structure of the gemstone is performed using a scanning optical projection tomography system (scanning-OPT configuration).

FIG. 4 is a flow chart 400 illustrating a specific embodiment of method 200 for determining one or more features associated with an internal structure of a gemstone. At step 402, a data acquisition process is performed, which encompasses the step 202 and 204 of method 200.

Steps 404 to 412 describe a specific embodiment of the processing step 206 of method 200.

At step 404, a model of an external surface geometry of the gemstone is determined.

At step 406, a model of a simulated homogeneous sample is generated, the simulated homogenous sample comprising a simulated external surface having the determined external surface geometry of the gemstone and wherein the simulated homogeneous sample has a homogeneous refractive index.

At step 408, a model is generated of a simulated propagation of simulated EM radiation between the source of incident EM radiation and the optical detecting means and via the simulated homogeneous sample, wherein a model of interaction of the simulated EM radiation with a plurality of virtual surface boundaries of the simulated homogeneous sample is generated. In one embodiment as described in the following, each virtual surface boundary will be understood to refer to a virtual surface boundary at the simulated external surface of the simulated homogeneous sample.

At step 410, an attenuation of simulated EM radiation is modelled using the model of simulated propagation including the model of interaction of the simulated EM radiation with the plurality of virtual surface boundaries of the simulated homogeneous sample.

At step 412, a 3D distribution of an optical property within the interior of the gemstone is reconstructed, the 3D distribution of the optical property being indicative of a 3D distribution of the one or more features in the internal structure of the gemstone.

### Step 402—Data Acquisition

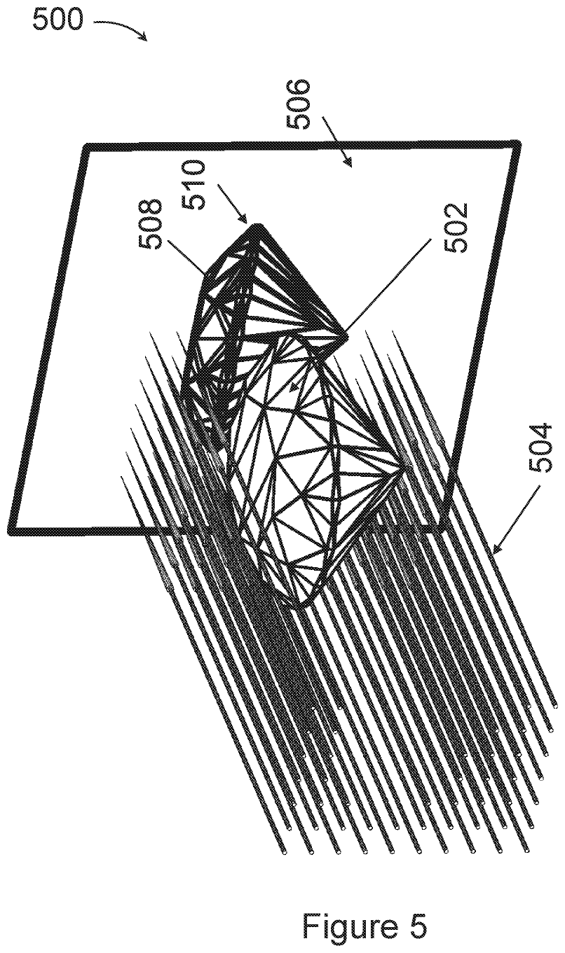

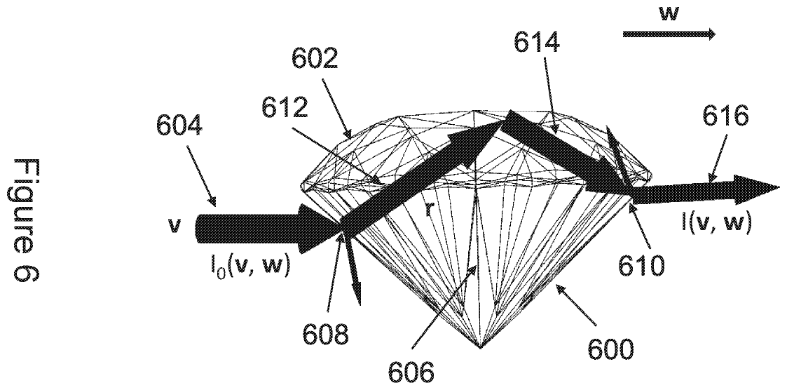

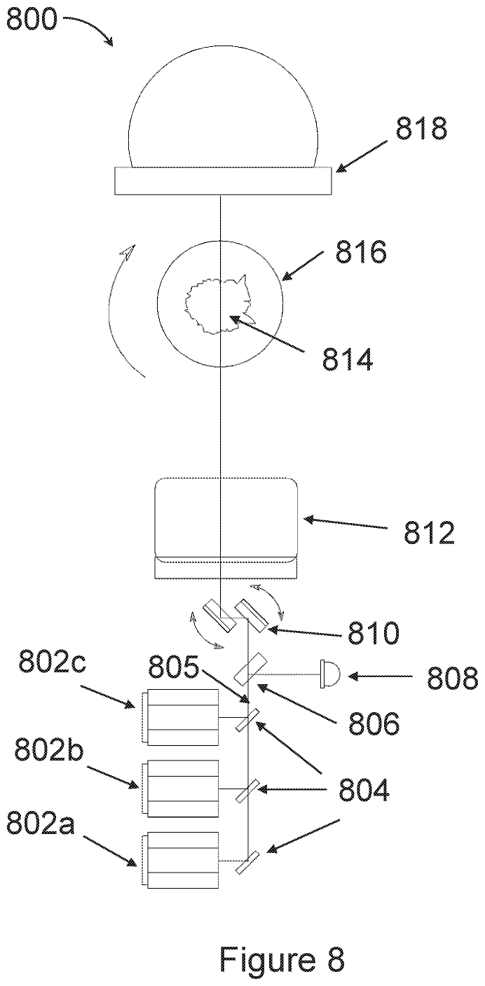

FIGS. 5 and 8 provide simplified example illustrations 500 and 800 of the data acquisition process 402, wherein steps 202 and 204 of method 200 are performed using a scanning approach, a scanning optical system 800 such as shown in FIG. 8 being used, which is similar to scanning optical system 300 shown in FIG. 3a.

With reference to FIG. 8, in a specific embodiment, the laser source of incident EM radiation 302 comprises three separate lasers 802a, 802b, 802c of three different wavelengths, which in the present specific embodiment are in the visible wavelength range. For example, the three different wavelengths may each be within the wavelength range 400-500 nm, 500-600 nm, 600-700 nm or any wavelength range with the visible wavelength range. The three beams from the separate lasers 802a, 802b, 802c are combined in an overlapping manner using dichroic mirrors 804. A fraction of the combined laser beam 805 is sampled using an uncoated glass plate beam sampler 806 and a photodetector 808 to serve as a reference measurement of power of incident EM radiation. The passage of the laser beam 805 through the uncoated glass plate beam sampler 806 may facilitate sampling of a fixed fraction of the incoming light power on a reference detector. This may be used at a later stage to remove laser power fluctuations from the resulting acquired data by dividing the power or intensity of the detected EM radiation by the reference measurement, yielding dimensionless attenuation, transmissivity or scattering strength ratio measurements. The laser beam 805 then passes through a galvanometer scanner 810 and a scan lens 812 to facilitate scanning of the laser beam in a controlled pattern across a gemstone 814, which is mounted at the working distance of the scan lens 812 on a rotation stage 816, which allows to change the relative alignment of optical system and gemstone 814 to acquire views from different directions. In one embodiment, the laser beam is scanned across the gemstone 814 in a rectangular grid pattern with fixed spacing between consecutive scan points and scan lines. However, a square pattern with parallel beam propagation may also be used, with fixed spacing between consecutive scan points and scan lines, and it will be understood that any geometry is envisaged, provided that the resulting light ray paths are known. The set of beams of electromagnetic radiation produced by the galvanometer scanner need not be parallel to one another, and under some circumstances non-parallel beam scanning might be desirable to improve coverage of the internal volume of the test sample or gemstone. Finally, a laser beam is detected by a large area photodetector 818, which is located as close as is practical to the gemstone 814 so as to detect as wide a range of ray paths exiting the gemstone 814 as possible. Such an arrangement may allow maximising the number of scans intersecting with the detector 818 after passing through the gemstone 814.

It may further contribute to improving the efficiency of the data acquisition as rays of EM radiation that miss the detector cannot be used to measure the amount of loss or scattering encountered along that path.

A computer is typically used to control the galvanometer scanner 810 inputs and thus the current scan direction, while simultaneously sampling the reference photodetector 808 and measurement photodetector 818. By computing the ratio of the respective measured EM radiation power of these two detectors 808 and 818, and correlating it with the corresponding scan position, a 3D map of the resulting transmissivity can be produced. This map is then stored for each view, i.e. each incident direction of the EM radiation and each laser wavelength, either independently or in a combined measurement file, which is later used for the reconstruction, using the processor, of the 3D distribution of one or more features associated with the internal structure of the gemstone.

In one embodiment, with reference to FIG. 5, the following is accounted for during the acquisition step.

In FIG. 5, the gemstone 502 is shown in the path of scanned laser beams 504. It is to be noted that as a simplification, only the laser beams 504 that do not interact with the gemstone surface or interior are shown. These laser beams 504 strike the detector (not shown) without attenuation due to interaction with the gemstone external surface and gemstone interior. For each scan point of the rectangular pattern, EM radiation is detected on the large area photodetector (such as photodetector 310 and 818) positioned on the other side of the gemstone substantially opposite the source of incident EM radiation, wherein an intensity of the detected EM radiation is measured and recorded. Although the laser beams interacting with the surface and interior of the gemstone 502 are not shown in FIG. 5, all EM radiation that interacts with the gemstone's external surface and propagates through the gemstone's interior undergoes some attenuation and the EM radiation that reaches the detector after these interactions with the gemstone's surface and interior is characterised by reduced intensity. An intensity of the incident EM radiation and the intensity of the detected EM radiation may then be used to determine a normalised attenuation of the EM radiation following a propagation of the EM radiation between the source of EM radiation and the optical detecting means and via the external surface and interior of the gemstone 502. Using the intensity of the detected EM radiation or the determined normalized attenuation of the EM radiation for each scan point of the rectangular pattern, a two-dimensional (2D) projection image can be obtained on a rectangular grid 506 in grey scale, which corresponds to a 2D representation or 2D projection view of the attenuation of EM radiation after propagation of the incident laser radiation through to the large area photodetector and via the gemstone 502.

The process is then repeated for different relative orientations of the gemstone and the source of incident EM radiation, i.e. the laser source. In a specific embodiment, the gemstone is rotated about one or more rotation axes that are centred on, and perpendicular to, the incident direction of the EM radiation from the laser source. Typically, 2D projection views are obtained and recorded for a large number of rotation steps, for example, however not limited to, more than 360 rotation steps.

In one embodiment, the detected EM radiation and obtained 2D projection views may be corrected for intensity fluctuations of the incident laser radiation. To do so, in one example, the separate reference photodetector 808 positioned in front of the galvanometer scanner 810 may be used to record intensity of EM radiation reflected by the uncoated glass plate beam sampler 806 positioned on the laser beams path, and the intensity of the reflected EM radiation may be used to correct for the intensity fluctuations of the incident laser radiation. Further, any variations in the spatial and angular sensitivity of the detector, as well as any temporal variations in the power of the lasers may be characterised and measured so as to correct the detected EM radiation and obtained 2D projection views for these variations. For example, to characterize variations in the spatial sensitivity of the detector and in the intensity of the incident laser radiation, an intensity of detected EM radiation may be recorded for an arrangement without a gemstone in the beam path (bright field) and be used at a later stage to correct the experimental data (i.e. data obtained with the gemstone in the beam path) for the mentioned variations.

It will be understood that the provided example implementation of the data acquisition is only one potential implementation and that embodiments of the present invention are not limited to this implementation, which is only used as an example.

### Step 404—Obtaining a Model of External Surface Geometry

The shape, i.e. geometry, of the external surface of the gemstone accounted for in the processing step 206 may be determined using different techniques.

In one embodiment, data associated with the external surface geometry of the gemstone is determined using the EM radiation detected at step 204, i.e. the data characteristic of the detected EM radiation such as the intensity of the detected EM radiation. In this embodiment, with reference to FIG. 5, as a result of the interaction of the EM radiation with the gemstone 502 external surface and interior, the gemstone 502 causes a dark shadow 508 with a silhouette 510 that can be determined through a standard image processing method such as thresholding. It will be understood that the shadow 508 illustrated in FIG. 5 is only a schematic representation. In practice, the dark shadow caused by the gemstone as a result of the interaction with the EM radiation generally comprises a range of grey shades. Further, using the 2D projection views obtained and recorded for the large number of rotation steps during the data acquisition process and using the determined corresponding silhouettes 510, an approximate 3D model (called a visual hull) of the external surface geometry of the gemstone 502 can be obtained using a 3D reconstruction technique or an iterative tomographic image reconstruction technique. A number of known algorithms associated with such techniques may be used, such as, e.g., the algorithm introduced by A. Laurentini (1994, IEEE Transactions on Pattern Analysis and Machine Intelligence. pp. 150-162). The visual hull three-dimensional reconstruction technique provides data indicative of the position and orientation of the gemstone and gemstone surface relative to the source of EM radiation and the optical detecting means.

In another embodiment, data associated with the external surface geometry of the gemstone is determined separately using one or more of a variety of known techniques such as, for example, X-ray computed tomography (XCT), optical surface tomography such as optical coherence tomography (OCT), or optical surface scanning. The determined data associated with the external surface geometry of the gemstone may then be used to process the EM radiation detected at step 204 and obtain information indicative of the one or more features associated with the internal structure of the gemstone. Using an XCT technique, a scan of the gemstone is acquired that allows obtaining a high-resolution 3D grayscale map of the entire gemstone, and from which a model of the external surface geometry can be extracted, e.g., using a computer graphics algorithm such as “Marching cubes”. Using an OCT technique, a plurality of scans of the gemstone are acquired for multiple viewing directions so as to capture scans substantially representative of the entire external surface of the gemstone. Like XCT, OCT generates a 3D grayscale dataset from which a surface model can be extracted, e.g., using a computer graphics algorithm such as, however not limited to, “Marching cubes”. Using an optical surface scanning technique, a 3D surface scanner is used to obtain the data associated with the external surface geometry of the gemstone such as a structured light 3D scanner or any scanning laser as considered appropriate by the person skilled in the art.

In a further embodiment, a consolidated determination of the external surface geometry of the gemstone is carried out by combining, using an algorithm, the visual hull data and the data obtained using a separate technique as mentioned above. Examples of such as techniques include (but are not limited to) XCT, optical surface tomography such as OCT, or optical surface scanning.

This embodiment may be particularly suitable for a gemstone having a rough irregular external surface. The processing of the visual hull data and the separate technique data in combination contributes to improving an accuracy of the determination of the gemstone's external surface geometry.

Subsequently, the determined external surface geometry of the gemstone is used for further processing of the detected EM radiation data so as to obtain information indicative of the one or more features associated with the internal structure of the gemstone.

The gemstone may not have the same orientation and position for each acquisition of, respectively, the data characteristic of the detected EM radiation (at step 204 of the method 200) and the separate technique data (for example using XCT, OCT or optical surface scanning as mentioned above).

To proceed with the reconstruction process and determine the one or more features associated with the internal structure of the gemstone, an orientation and position of the gemstone during acquisition of the data for determining the external surface geometry of the gemstone and an orientation and position of the gemstone during acquisition of the data characteristic of the detected EM radiation at step 204 of the method 200 must be taken into account. Coordinates indicative of the orientation and position of the gemstone 502 relative to the scanning optical system 300 when acquiring the data characteristic of the EM radiation detected at step 204 (referred to in the following as “projection data”) need to be “aligned” with coordinates indicative of the orientation and position of the gemstone 502 relative to the data acquisition system of the separate technique (referred to in the following as ‘external surface geometry data’). The external surface geometry data and the projection data may be aligned by several approaches: (a) physical alignment of the two optical scanning systems used for the acquisition of, respectively, the external surface geometry data and the projection data (e.g. by integrating both scanning systems into a single apparatus); (b) using registration marks on the gemstone; or (c) creating an approximate external surface geometry model from the projection data using the visual hull reconstruction technique as described above, and then use a 3D registration method to align the obtained visual hull to the external surface geometry model obtained separately (referred to in the following as “separate external surface geometry model”) using one of the techniques such as, however not limited to, XCT, optical surface tomography such as OCT, or optical surface scanning. This latter aligning approach (c) will now be further described.

The orientation of the separate external surface geometry model relative to the visual hull model is determined by determining the relative translation, scale and rotation of one of the models (i.e. the separate external surface geometry model or the visual hull model) that aligns the one model most closely with the other one of the models (i.e. the other one of the separate external surface geometry model and the visual hull model). Such determination may be performed, in a standard case, using a technique known as mesh registration where both models are represented as a surface mesh. Any known mesh registration technique as considered appropriate by the person skilled in the art may be used.

Using the determined external surface geometry of the gemstone, reflection and refraction of rays of EM radiation at the external surface of the gemstone can then be modelled, whereby refraction and reflection effects due to the gemstone external surface geometry can be accounted for to process the detected EM radiation. Modelling the refraction and reflection effects due to the external surface geometry of the gemstone enables a compensation for these effects during the analysis/processing of the detected EM radiation to determine features internal to the gemstone. Thus, avoiding the need to envelop the gemstone in a refractive index matching material.

Modelling of a simulated propagation of simulated electromagnetic radiation between the source of incident electromagnetic radiation and the optical detecting means and via a simulated homogeneous sample will now be described, with reference to steps 406 and 408 of flow chart 400.

### Steps 406 and 408—Modelling a Simulated Interaction of Simulated EM Radiation with a Plurality of Virtual Surface Boundaries of a Simulated Homogeneous Sample Having a Simulated External Surface with a Geometry that Corresponds to the Determined External Surface Geometry of the Gemstone and Having a Homogeneous Refractive Index

In one embodiment, an assumption is made that the source of simulated EM radiation is quasi-monochromatic (or may consist of multiple independent quasi-monochromatic components), where quasi-monochromatic means that the EM radiation has a sufficiently narrow spectrum such that gemstone properties do not vary significantly within that spectrum. Further, another assumption is made that after simulated refraction of the simulated EM radiation at one or more virtual boundaries of the simulated homogeneous sample, the simulated EM radiation propagates within the simulated homogenous sample without refraction.

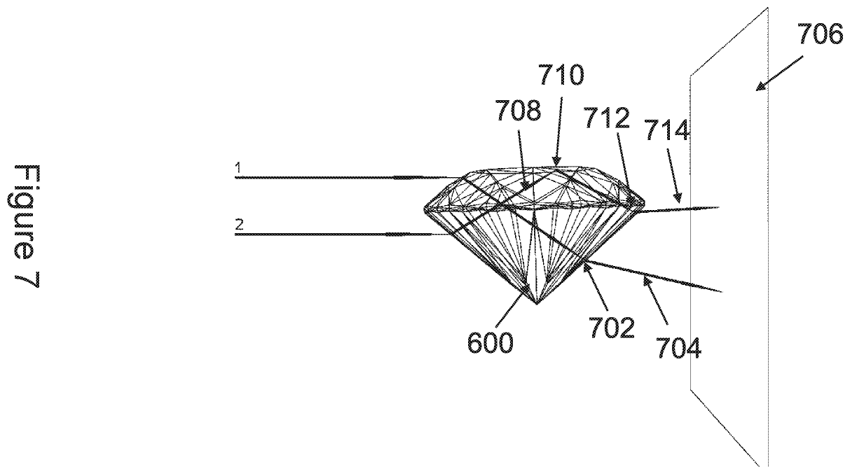

With reference to FIG. 6, a simulated homogeneous sample 600 is represented having a simulated external surface 602 with a geometry corresponding to the external surface geometry of the gemstone 502 determined at step 404. Quasi-monochromatic EM radiation 604 is simulated with intensity I₀(v, w) originating from point v, travelling in direction w. For each ray (v, w), a simulated total attenuation of each ray of EM radiation is modelled as the product of the attenuation due to the simulated internal structure of the simulated homogeneous sample 600 and the attenuation due to the simulated homogeneous sample 600 external surface 602 (for clarity, we note that this product is a sum when expressed in logarithms of attenuation, as is done in the description of Step 410 below).

In order to model propagation of EM radiation within a highly-refracting gemstone, both the following elements are calculated: (i) the geometry of the path that each incident ray of EM radiation follows within a simulated interior 606 of the simulated homogeneous sample 600, and (ii) an intensity loss in the EM radiation as the EM radiation is transmitted through and reflected from the virtual surface boundaries 608 and 610 at the simulated external surface of the simulated homogeneous sample 600. For elements (i) and (ii), the following mathematical notations are introduced:
1. A set of ray-path segments R(v, w), corresponding to a set of line segment(s) r within the simulated homogeneous sample 600 traversed by simulated EM radiation with an original incident direction w and origin v. Each segment r may be defined by its start and end points (x₁, x₂) but may also be defined by its start point, direction and length, (v′, w′, I′), where the symbol “′” indicates interior rays.
2. A function s[R(v, w), p] that is the total attenuation of the ray due to Fresnel reflection at a surface boundary of the simulated homogeneous sample 600 for simulated EM radiation having a simulated incident polarisation state p. Attenuation is defined as the ratio between the incident and transmitted light, i.e. s =Iₛ/I₀ where I₀ is the incident intensity and Iₛ is the intensity that would emerge from the simulated homogenous sample 600 being completely transparent with a homogeneous refractive index.

The parentheses/brackets after s and R in the above denote a functional dependence: intensity loss at the surface depends on the ray-path and the initial polarisation state, which in turn depend on the direction and origin-point of the beam of simulated EM radiation. If the incident polarisation state p is not uniform, the function s would depend on the ray direction and origin and be written as P(v, w). It is noted that the term s[R(v, w), p] for modelling interactions with surface boundaries of the sample is only well defined if the initial polarisation state p of the light beam is known.

Further, it is noted that R and s model the paths and the intensities of the simulated EM radiation in the case of a simulated homogeneous sample 600 that is transparent and has a homogeneous refractive index. In one embodiment, it is assumed that any structure within the simulated homogeneous sample 600 affects the ray's intensity but not its path, and accordingly the modelled ray paths of the simulated EM radiation, the simulated polarisation state and the simulated surface attenuation do not change in the presence of an attenuating internal structure in the simulated homogeneous sample 600.

Both R and s can be calculated for each ray (defined by v and w) as long as the simulated polarisation state p is known, using the external surface geometry model determined at step 404, ray tracing, Snell's law and Fresnel equations (reproduced in equations (1) and (2) below) along with the simulated homogeneous sample's homogeneous refractive index.

$$Snell ’ s law : \frac{sin ( {θ}_{1} )}{sin ( {θ}_{2} )} = \frac{{n}_{2}}{{n}_{1}} \qquad ( 1 )$$

$$Fresnel equations : {F}_{R , ‖} = \frac{{n}_{2} cos {θ}_{1} - {n}_{1} cos {θ}_{2}}{{n}_{2} cos {θ}_{1} + {n}_{1} cos {θ}_{2}} , {F}_{R , ⊥} = \frac{{n}_{1} cos {θ}_{2} - {n}_{2} cos {θ}_{1}}{{n}_{1} cos {θ}_{2} + {n}_{2} cos {θ}_{1}} \qquad ( 2 )$$

In a more general case where a sample would alternatively be simulated as being in-homogeneously birefringent, surface attenuation would not be known directly from the measured projection data but would depend on the simulated internal structure of the simulated sample. This case requires a more complex processing formulation where surface attenuation is included in the modelling for the reconstruction step 408.

In one embodiment, the transverse beam shape (profile) is also modelled to better capture the simulated interactions of simulated EM radiation with virtual surface boundaries of the simulated homogeneous sample. An accurate model of the beam shape can be used to model depth-of-field effects. Once this model is incorporated into the simulation, features outside the depth-of-field may appear diffuse (or entirely absent). These diffuse features may be down-weighted (or ignored entirely) in a correction step. These weights may be hard-coded. For example, the features outside the depth-of-field may be ignored entirely, or they may be calculated.

### Ray Tracing

Propagation of the simulated EM radiation is in one embodiment simulated using a simplified ray-optics model.

A path of each simulated beam of simulated EM radiation is traced in segments. First, the simulated beam is traced from the position v in the direction w until the simulated beam either: (i) intersects the detector (not shown) and is recorded without losing intensity; (ii) misses the simulated homogeneous sample and detector, in which case the corresponding intensity measurement contains no information and is removed from the data set; or (iii) intersects the simulated external surface of the simulated homogeneous sample (where it may or may not ultimately intersect the detector). Where the simulated beam intersects the simulated external surface of the simulated homogeneous sample, a simulated refraction of the simulated EM radiation as well as a simulated reflection of the simulated EM radiation at one or more virtual surface boundaries of the simulated homogeneous sample are then modelled.

### Ray-Surface Interactions

When a traced ray intersects the simulated external surface of the simulated homogeneous sample, it will do so at a point. However, the beam of EM radiation is of a finite width (transverse size), and so will interact with the simulated external surface of the simulated homogeneous sample in a finite-sized patch around this point. Known properties of the EM radiation are used to determine the size of this region of interaction (RoI). In one embodiment, the transverse profile of the simulated beam of simulated EM radiation is modelled as a Gaussian beam and the known properties may be the width and position of the beam waist of the Gaussian beam. However, it will be understood that embodiments of the present invention are not limited to the simulated beam being a Gaussian beam and other numerical models of beam propagation may be used, such as for example a Bessel beam.

To simulate the interaction between simulated EM radiation and the simulated external surface of the simulated homogeneous sample within this RoI, the simulated beam of simulated EM radiation is temporarily split into a plurality of simulated rays of simulated EM radiation, each ray obeying the ray-optics approximation, and each ray being incident on a different part of the RoI. It is to be noted that if the simulated beam isn't considered to have a finite -width then the simulation of the interaction between simulated EM radiation and the simulated external surface of the simulated homogeneous sample within the RoI is applied with just a single ray of simulated EM radiation.

Ray-tracing is then performed for each simulated ray to calculate the angle-of-incidence and point-of-interaction at virtual surface boundaries at the simulated external surface of the simulated homogeneous sample. For each simulated ray within the simulated beam of simulated EM radiation, Snell's law is used to calculate the angle of refraction, while the Fresnel equations are used to calculate the intensity of reflection and refraction for the “s” and “p” simulated polarisation components.

Information obtained from the Fresnel equations can be summarised in a Mueller matrix, which allows the polarisation state and intensity of the EM radiation detected at the detector to be simulated following surface interactions at the virtual surface boundaries of the simulated homogeneous sample.

With reference to FIG. 6, an initial simulated ray 604 of 100% intensity is traced to its first intersection point at virtual boundary 608, where it refracts and partially reflects due to Fresnel reflection, leading to a loss of intensity in the simulated beam 612 of simulated EM radiation refracted at and transmitted through the surface of the simulated homogeneous sample 600, which refracted simulated beam 612 is further traced. At the next surface intersection point at 614, the simulated ray 612 undergoes total internal reflection. No further intensity is lost at this point, but the simulated reflected ray 614 is traced to the next intersection point with the surface at 610, at which point the simulated ray 614 refracts again with simulated refracted ray 616 being transmitted and part of the simulated EM radiation is also reflected again, leading to another loss of intensity.

If the Mueller matrices and angles-of-refraction for all simulated rays within a simulated beam (v, w) of simulated EM radiation are sufficiently similar, a simulation of the beam of simulated EM radiation can further be conducted. To do so, a weighted average is calculated of the Mueller matrices and angles-of-refraction for each simulated ray within the simulated beam of EM radiation. Weightings are determined according to the transverse intensity profile of the simulated beam of simulated EM radiation. If Mueller matrices and angles-of-refraction for all simulated rays within a simulated beam of simulated EM radiation are not sufficiently similar and further simulation of the beam of simulated EM radiation cannot be conducted, a measurement of the detected simulated EM radiation corresponding to the traced simulated beam of simulated EM radiation is considered to not contain useful information and is removed from the dataset.

Thus, an averaged angle-of-refraction determined for the simulated ray 604 of simulated EM radiation as it enters the gemstone 600, in combination with the location where it intersects the surface of the gemstone 600 at 608, provides information for ray-tracing along a new ray-path segment beyond the surface of the gemstone 600. If that new segment is within the gemstone 600, then it is added to the set R(v, w). The resulting average Mueller matrix describes changes to the beam polarisation state and intensity and is generated for each ray-surface interaction. The intensity change is recorded to s[R(v, w), p] and the new polarisation state of the beam recorded.

The above-described test for uniformity of all simulated rays within a beam (v, w) is applied at each location where the beam interacts with the surface—i.e. at the exit location and at any locations of partial or total internal reflection along the beam path. If the Mueller matrices and angles-of-refraction for all simulated rays within a simulated beam of simulated EM radiation are not sufficiently similar at any of these locations, then the data for this beam is removed from the dataset.

### Tracing to the Detector

Using the new direction of propagation and the aforementioned beam properties, the process is repeated to determine a second region-of-interaction (RoI) between the surface of the gemstone 600 and the simulated beam of simulated EM radiation. This time, the simulated beam is striking a virtual surface boundary of the gemstone 600 from the interior. For the modelling, i.e. ray tracing model, virtual surface boundaries are virtual surface boundaries at the simulated external surface (corresponding to the determined external surface geometry of the gemstone) of the simulated homogeneous sample. The second region-of-interaction is modelled in the same manner as the first interaction, however there are two distinct cases to consider which is illustrated in FIG. 7. In a first case, if the simulated beam strikes the virtual surface boundary 702 of the gemstone 600 with an angle of incidence of less than the critical angle for the material of the gemstone 600 without total internal reflection, as depicted for ray number 1 in FIG. 7, the simulated beam exits, the ray-tracing simulation of the simulated ray segment 704 refracted in the exit direction is conducted to determine if the refracted simulated ray segment 704 eventually hits the detector at 706. If the simulated beam or ray segment 708 entering the interior of the gemstone 600 undergoes total internal reflection at surface boundary 710, as depicted for ray number 2 in FIG. 7, new simulated ray segments are traced, and added to the set R(v, w) until the simulated beam finally strikes the exit surface 712 with an angle of incidence below the critical angle and at least partially exits the gemstone 600. When the simulated beam eventually exits the gemstone 600, a respective simulated exiting ray segment 714 is traced to see whether the simulated exiting ray segment hits the detector at 706. If it does, the intensity change is recorded to s[R(v, w), p] and the new polarisation state of the beam is recorded. If it does not, the measurement once again contains no useful information, and is removed accordingly.

It is noted that it is also envisaged to further model the paths of reflected portions of each simulated ray of simulated EM radiation to see whether the reflected portions eventually hit the detector, in which case the simulated intensity of simulated EM radiation reaching the detector would be the result of multiple interactions.

It is noted that iterative analysis may be used to map the simulated ray paths attributable to each internal boundary of the gemstone. For ray-tracing modelling within the interior of the gemstone, a threshold number of total simulated internal reflections, e.g. 1 or 2, may be considered to minimise uncertainties with each ray direction change. It should be appreciated that the accuracy of the ray tracing modelling may degrade where multiple virtual surface boundaries are encountered by the simulated beam of EM radiation, and the ray tracing modelling may be less reliable the more virtual surface boundaries are encountered.

### Step 410—Modelling the Attenuation of EM Radiation Using the Ray-Tracing Modelling

Following the data acquisition steps 402 and 404 and as a result of the above simulations 406 and 408, for each simulated ray (v, w) in the scan, the following elements of input information are obtained for further processing by a processor:
experimental data for the simulated ray's incident and transmitted intensity (and polarisation state p)
a model that includes, for each simulated ray:
   the path that it traverses through the sample interior: R(v, w); and
   the attenuation that it suffers as a consequence of at least two interactions with the sample surface: s[R(v, w), p]

As shall be described in the following, it is possible, using standard methods of computed tomography, to configure the processor to process the above elements of input information to generate an output associated with a linear attenuation coefficient of the internal structure of the test sample.

Comparison with Conventional Tomography

In conventional tomographic imaging or computed tomography, measurements are collected that correspond to sums over straight rays (i.e. line integrals) through some 3D quantity of interest. In other words, rays of EM radiation move through the sample in straight lines and accumulate changes (e.g. attenuation) as they go. In a conventional tomographic imaging setup, it is assumed that refraction is negligible and that the ray paths are straight lines.

The modelling of propagation of the EM radiation in accordance with embodiments of the present invention differ from conventional computed tomography in several important ways:
1. In accordance with embodiments of the present invention, significant refraction can occur at the surface of the gemstone or test sample (see e.g. FIG. 6 or FIG. 7). This alters the direction of propagation of the EM radiation, so that the beam of EM radiation no longer propagates in its original direction w. A set of ray-path segments R(v, w), corresponding to the line segment(s) r within the gemstone 600 traversed by EM radiation with an incident direction w and origin v is thus considered.
2. The set of data R(v, w) may contain more than one straight-line element. This occurs when instead of exiting the gemstone, EM radiation undergoes total internal reflection at a surface boundary of the gemstone. Measurement of the intensity of transmitted EM radiation, when it does eventually exit the gemstone and hits the detector, corresponds to line integrals over multiple connected line segments within the gemstone. In conventional tomography however, each ray travels in a single direction without deviation.
3. In general, EM radiation that intersects surface boundaries of the gemstone loses some of its intensity due to Fresnel reflection. The amount of intensity lost is described by the Fresnel equations and depends on the polarisation state of the EM radiation. The polarisation state of the light is described using a Stokes vector.
   Mapping the Modelling in Accordance to Embodiments of the Present Invention onto Conventional Tomography

In conventional tomography of non-refracting test samples, the model of propagation of EM radiation is referred to as the “X-ray projection transform” operator [symbol] that predicts the total attenuation that will be suffered by each ray (v, w) that passes through a volume with a spatial distribution of attenuation μ(x), i.e.:

[symbol]

[μ(x), (v, w)]=−ln[I(v, w)/I₀(v, w)] (3)
where x is a 3d cartesian coordinate system anchored to the test sample.

Mathematically, in accordance with an embodiment of the present invention, the model of propagation of the EM radiation is defined as a counterpart to the projection operator (i.e. model of propagation) that is used in conventional computed tomography. The “generalised projection operator” in accordance with embodiments of the present invention comprises two terms:

[symbol]

′[μ(x), (v ,w)]≡ [symbol] _{R}[μ(x), (v, w)]+ln(s[R(v, w), p]), (4)

The quantities [symbol] _{R}[μ(x), (v, w)] and ln(s[R(v, w), p]) correspond to two parts of the model:
1. [symbol] _{R}[μ(x), (v, w)] corresponds to a modelling of a loss of intensity as the EM radiation propagates through the interior of the gemstone, due to internal features (e.g., however not limited to, inclusions). [symbol] _{R}[μ(x), (v, w)] is the sum of the 3D linear attenuation coefficient (a map of how attenuating the gemstone is) along the ray-path segments R(v, w);
2. ln(s[R(v, w), p]) corresponds to a model of a loss of intensity in the EM radiation as a result of interaction of the EM radiation at virtual surface boundaries of the gemstone. “s” is a function describing the intensity loss due to Fresnel reflection at the one or more virtual surface boundaries, for EM radiation having an incident polarisation state p.

This generalised projection operator captures behaviours important to the optical imaging system whilst retaining important mathematical properties of the more conventional projection operator [symbol] .

### Step 412—Obtaining a Reconstruction of a 3D Distribution of an Optical Property Within the Interior of the Gemstone

Using as inputs: (i) the detected EM radiation (as at step 204 of method 200) and (ii) the generalised projection operator [symbol] ′ (a non-linear integral transform modelling the propagation of EM radiation, which comprises modelling attenuation of the EM radiation as it propagates between the source of incident EM radiation and the detector and via the gemstone), the processor is arranged to generate an output associated with a three-dimensional distribution of an optical property within the internal structure of the gemstone, the optical property being associated with an interaction between the gemstone and the incident electromagnetic radiation directed from the respective incident direction, the three-dimensional distribution of the optical property being indicative of a three-dimensional distribution of the one or more features (including defects, inclusions, impurities, chromatic properties, polarisation properties) in the internal structure of the gemstone. For example, using an optical projection tomography system for data acquisition, the optical property may be the transmissivity. In another embodiment where EM radiation scattered from within the gemstone is detected, the optical property may be the scattering strength. The optical property is typically related to the intensity of the EM radiation detected at the detector/sensor.

The three-dimensional distribution of the optical property is indicative of a three-dimensional distribution of the one or more features within the internal structure of the gemstone and the processor is arranged to determine the one or more features associated with the internal structure of the gemstone using the determined three-dimensional distribution of the optical property.

### Background

The conventional tomographic reconstruction problem of calculating a 3D quantity of interest, such as a 3D linear attenuation coefficient p(x), from its line integrals [symbol] [μ(x), (v, w)] is equivalent to calculating an inverse to [symbol] . For sufficiently straightforward tomographic imaging experiments, this is done using “Fourier-slice” based methods, or “filtered back-projection”.

In the more general case, calculation of the inverse operator is either unfeasible or impossible. If so, a solution is iteratively approximated using algorithms such as SIRT, SART, or EMTR. These algorithms run (iterate) multiple times, each time producing a result progressively closer to the correct answer. Furthermore, these algorithms have the significant advantage that they do not require knowledge (or the existence) of the inverse operator.

### Using Existing Iterative Reconstruction Algorithms

Once a generalised projection operator [symbol] ′ is defined and the associated modelling completed (yielding simulated ray paths R(v, w) and attenuations s[R(v, w), p], it is straightforward to use well-established algorithms to reconstruct the 3D linear attenuation coefficient μ(x) from the recorded intensities I(v, w) and I₀(v, w) contained in the projection data. To do so, a generalised back projection operator [symbol] ′ is defined as the adjoint of [symbol] _{R}. This operator denotes back projection (see, for example, A. C. Kak and Malcolm Slaney, Principles of Computerized Tomographic Imaging, Society of Industrial and Applied Mathematics, 2001) along the ray-path segments R(v, w). Back projection is a term used in computed tomography, which can be described colloquially as the “smearing” of output values along the corresponding ray-path segments.

To solve for μ(x), an algorithm such as SIRT or EMTR may be used, which may be found in publications from the literature relating to tomography (e.g. for EMTR, Kenneth Lange, Richard Carson, et al., Em reconstruction algorithms for emission and transmission tomography, Journal of Computer Assisted Tomography 8(2):306-16, 1984; for SIRT, A. C. Kak and Malcolm Slaney, Principles of Computerized Tomographic Imaging, Society of Industrial and Applied Mathematics, 2001), in which the conventional projection and back projection operators are replaced with the generalised variants described above. The algorithm is then run until it converges on a result. In certain cases, e.g., where it is known that the gemstone is predominantly transparent with only isolated defects, compressed-sensing algorithms that assume spatial sparsity (such as CS-SIRT) may be particularly useful. Poisson or Gaussian noise at the detector may also be taken into account.

As mentioned previously, the term s[R(v, w), p] for modelling interactions with surface boundaries of the sample is only well defined if the initial polarisation state p of the light beam is known. If the linear attenuation coefficient μ(x) is known, the initial polarisation state of the incident EM radiation may be determined by searching for the set of parameters which best matched the input data:

p(v, w)=argmin[p′(v, w); |−ln[I(v, w)/I₀(v, w)]− [symbol] _{R}[μ(x), (v, w)]+ln(s[R(v, w), p′(v, w)])|_{0.5}], (5)

where argmin[a; b(a)] is the value of a that minimises the function b(a), and |.|_{0.5} is the p-norm for p=0.5. In one embodiment, the p=0.5 norm is used, which emphasises matching broad/flat features in images, but another norm may alternatively be used.

In practice, neither p(v, w) or μ(x) is known exactly. However, one of these parameters can be approximated with an estimate of the other using the following iterative procedure:
1. Begin with estimates p⁽⁰⁾(v, w), and μ⁽⁰⁾(x) at iteration 0. It is reasonable to begin with unpolarised light, and an empty (transparent) object;
2. Solve for a new estimate for the polarisation:

p^{(t)}(v, w)=argmin[p′(v, w); |−ln[I(v, w)/I₀(v, w)]−P_{R[μ}^{(t-1)}(x), (v, w)]+ln(s[R(v, w)])|_{0.5}]; (6)
3. Given that estimate p^{(t)}(v, w), define a generalised projection operator and solve for a new estimate μ^{(t)}(x) to the linear attenuation coefficient, using an iterative tomographic reconstruction algorithm as described above;
4. Return to step 2 and repeat until convergence.

In accordance with an embodiment of the present invention, a “polarisation state” is defined using the Stokes parameters. These parameters are properties of the probability distribution of EM field states, so the above iterative procedure may be interpreted as an expectation-maximisation algorithm.

It is noted that in one embodiment, to obtain information associated with chromatic properties of the internal structure of the gemstone, a source of incident EM radiation emitting at multiple wavelengths, either one or multiple wavelengths-at-a-time, is used in step 402. For example, multiple wavelengths, one or multiple wavelengths-at-a-time, may be used for embodiments aiming at obtaining: (a) a map of the average colour (wavelength dependence) of the bulk test sample and/or of individual defects, and/or (b) an improvement of the illumination coverage within the test sample by varying the refraction angles of the light. In this case where multiple wavelengths are used in step 402, then one may reconstruct multiple 3D distributions of the optical property (e.g. one for each wavelength), and the complementary information from these 3D distributions may be combined using an algorithm to derive chromatic information about the one or more features associated with the internal structure of the gemstone.

Further, it will be understood that other properties of the source and beam of incident EM radiation may be modelled and taken into account by a processor for ray-tracing modelling and processing of the detected EM radiation. For example, other properties of the source and beam of incident EM radiation may include the beam size, focal plane and depth of field (for Gaussian beams, as other beam shapes may be characterised by different information), wavelength, location and direction.

Using iterative reconstruction techniques of the types described above, it is then possible to combine the optical property data determined by the processor for the different incident directions of the electromagnetic radiation to reconstruct the three-dimensional distribution of the optical property within the internal structure of the gemstone. The three-dimensional distribution of the optical property within the internal structure of the gemstone is indicative of a three-dimensional distribution of one or more features, such as inclusions and other defects, chromatic properties associated with the internal structure of the gemstone.

In one embodiment, the method may further comprise generating a three-dimensional graphical representation indicative of the three-dimensional distribution of the determined one or more features (including any one or more of the following: defects; inclusions; impurities; chromatic properties such as average colour, continuous variation of colour within the test sample, or average colour of inclusions/defects; polarisation properties) associated with the internal structure of the gemstone. In other words, the three-dimensional graphical representation corresponds to a three-dimensional reconstruction of one or more features within the internal structure of the gemstone. In this embodiment, the processor may be arranged to use the reconstructed 3D distribution of the one or more features associated with the interior of the gemstone to generate a corresponding 3D graphical representation.

In a further embodiment, it will be understood that it is envisaged to use an imaging approach instead of a scanning approach. In such an embodiment, a telecentric imaging system with a position-sensitive 2D imaging detector may be used. Instead of each measurement being associated with the EM radiation's incident direction w and point of origin v, each measurement will be associated with a position v on the detector and a vector w parallel to the optical axis of the telecentric lens. With this re-definition, ray-tracing begins at the detector instead of the source and the generalised projection operator is otherwise identical to that discussed above. The processor may then be configured to generate a model of simulated ray paths of simulated EM radiation between each image sensor pixel and the diffuse light source, wherein the imaging approach is the optical reciprocal to the scanning approach. More specifically, for each viewing angle, which relates to each pixel on the image sensor, the processor is configured to determine the ray exit point from the simulated homogeneous sample surface.

In cases where information associated with chromatic properties of the gemstone (e.g., bulk colour, or colour of inclusions) is to be extracted, transmitted intensity of EM radiation through the gemstone is measured for several different colour (wavelengths) of quasi-monochromatic light. Each of these measurements is reconstructed separately, resulting in respective 3D attenuation maps of the gemstone for each different colour. These are then combined using an algorithm to extract the information associated with chromatic properties of the gemstone (e.g., bulk colour, or colour of inclusions). In other words, the processor is arranged to determine optical property data for each wavelength (or narrow band of wavelengths) and a corresponding reconstruction of the three-dimensional distribution of the optical property within the internal structure of the gemstone is performed independently for each wavelength. Indeed, for each wavelength, the gemstone may yield different ray paths. The processor is arranged to determine chromatic properties associated with the internal structure of the gemstone (e.g., a colour of an inclusion or other defect; an average colour within the internal structure of the test sample; a continuous variation in colour in the internal structure of the test sample; a colour or average colour of an inclusion or other defect in the internal structure of the test sample; a brightness of an inclusion or other defect; an average brightness associated with the internal structure of the test sample; a fluorescence of an inclusion or other defect; an average fluorescence associated with the internal structure of the test sample) by accounting for differences between the various optical property data determined at the different wavelengths.

Embodiments of the present invention thus provide an advantage that a refraction-corrected reconstruction of a three-dimensional map of chromatic properties within the envelope of a sample, such as a gemstone, including one or more of the following: a colour of an inclusion or other defect; an average colour within the internal structure of the test sample; a continuous variation in colour in the internal structure of the test sample; a colour or average colour of an inclusion or other defect in the internal structure of the test sample; a brightness of an inclusion or other defect; an average brightness associated with the internal structure of the test sample; a fluorescence of an inclusion or other defect; and/or an average fluorescence associated with the internal structure of the test sample, may be determined without the need for submerging the test sample in different refractive index matching fluids or embedding the test sample in different refractive index matching solids.

More comprehensive generalisations of the above models may further be required for embodiments wherein the gemstone or other test sample is either homogeneously or in-homogeneously birefringent.

When detecting and using electromagnetic radiation scattered, reflected, and/or caused by fluorescence from within the test sample to identify defects or imperfections within the test sample, the source of electromagnetic radiation and the optical detecting means, i.e. detector or sensor, may be positioned by placing the detector to one side, or adjacent to the source.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features in various embodiments of the invention.

Modifications and variations as would be apparent to a skilled addressee are determined to be within the scope of the present invention.

It is also to be understood that, if any prior art publication is referred to herein, such reference does not constitute an admission that the publication forms a part of the common general knowledge in the art, in Australia or any other country.

1.-29. (canceled)

30. A method for determining one or more features associated with an internal structure of a gemstone, the method comprising:
directing electromagnetic radiation towards the gemstone using a source of incident electromagnetic radiation;
in response to directing electromagnetic radiation, detecting electromagnetic radiation using an optical detecting means, including detecting electromagnetic radiation following an interaction between the gemstone and the incident electromagnetic radiation; and
processing the detected electromagnetic radiation, wherein the processing:
accounts for a determination of an external surface geometry of the gemstone and for refraction and reflection effects due to the external surface geometry of the gemstone; and
obtains information indicative of the one or more features associated with the internal structure of the gemstone.

31. The method of claim 30, further comprising determining the external surface geometry of the gemstone.

32. The method of claim 30, wherein processing the detected electromagnetic radiation comprises determining the external surface geometry of the gemstone using the detected electromagnetic radiation.

33. The method of claim 30, wherein directing electromagnetic radiation towards the gemstone comprises directing electromagnetic radiation towards the gemstone from a plurality of different incident directions relative to the external surface geometry of the gemstone.

34. The method of claim 33, wherein detecting electromagnetic radiation comprises detecting electromagnetic radiation for each incident direction.

35. The method of claim 30, wherein processing the detected electromagnetic radiation comprises generating an output associated with a three-dimensional distribution of an optical property within the gemstone, the three-dimensional distribution of the optical property being indicative of a three-dimensional distribution of the one or more features associated with the internal structure of the gemstone.

36. The method of claim 35, wherein the method further comprises using the output to generate a three-dimensional graphical representation of the three-dimensional distribution of the one or more features associated with the internal structure of the gemstone.

37. The method of claim 30, wherein processing the detected electromagnetic radiation comprises generating, based on the determined external surface geometry, a model of a simulated propagation of simulated electromagnetic radiation between the source of incident electromagnetic radiation and the optical detecting means and via a simulated homogeneous sample, wherein interaction between the simulated electromagnetic radiation and the simulated homogeneous sample is accounted for, the simulated homogenous sample comprising a simulated external surface having the determined external surface geometry of the gemstone and wherein the simulated homogeneous sample has a homogeneous refractive index.

38. The method of claim 37, wherein directing electromagnetic radiation towards the gemstone comprises directing electromagnetic radiation towards the gemstone from a plurality of different incident directions relative to the external surface geometry of the gemstone, wherein detecting electromagnetic radiation comprises detecting electromagnetic radiation for each incident direction, and wherein the model of simulated propagation of the simulated electromagnetic radiation is generated for each incident direction.

39. The method of claim 37, wherein the method comprises modelling simulated refraction and attenuation of the simulated electromagnetic radiation at a plurality of virtual surface boundaries of the simulated homogenous sample.

40. The method of claim 37, further comprising modelling, based on the model of the simulated propagation, a simulated polarisation state of the simulated electromagnetic radiation propagating between the source of incident electromagnetic radiation and the optical detecting means and via the simulated homogeneous sample, wherein modelling the simulated polarisation state accounts for an interaction between the simulated electromagnetic radiation and respective virtual surface boundaries at the simulated external surface of the simulated homogeneous sample.

41. The method of claim 37, wherein the method further comprises modelling a shape and an intensity of a simulated beam of incident electromagnetic radiation, and wherein the method comprises using the modelled shape and intensity of the simulated beam of incident electromagnetic radiation to determine a size of a region of interaction of the simulated beam of incident electromagnetic radiation with virtual external surface boundaries of the simulated homogeneous sample.

42. The method of claim 30, wherein the one or more features include at least one or more of the following: a defect; an inclusion; an impurity; a chromatic property; a polarisation property.

43. The method of claim 30, wherein the method comprises directing electromagnetic radiation towards the gemstone at a minimum of two different wavelengths, wherein detecting electromagnetic radiation comprises detecting electromagnetic radiation for each wavelength, and wherein processing the detected electromagnetic radiation comprises processing the detected electromagnetic radiation for at least two wavelengths, wherein information indicative of one or more chromatic properties associated with the internal structure of the gemstone can be obtained.

44. The method of claim 30, wherein detecting electromagnetic radiation comprises detecting electromagnetic radiation scattered and/or reflected, and/or caused by fluorescence, from within the gemstone.

45. A system for determining one or more features associated with an internal structure of a gemstone, the system comprising:
a source of incident electromagnetic radiation configured to emit electromagnetic radiation towards the gemstone;
an optical detecting means configured to detect electromagnetic radiation including electromagnetic radiation following an interaction between the gemstone and the incident electromagnetic radiation; and
a processor configured to:
receive a first input associated with the detected electromagnetic radiation;
receive a second input associated with an external surface geometry of the gemstone; and
generate an output indicative of a three-dimensional distribution of an optical property within the internal structure of the gemstone, the optical property being associated with an interaction between the gemstone and the incident electromagnetic radiation, the three- dimensional distribution of the optical property being indicative of a three-dimensional distribution of the one or more features within the internal structure of the gemstone;
wherein the output is generated based on the first input, the second input, and accounting for refraction and reflection effects due to the external surface geometry of the gemstone.

46. The system of claim 45, wherein the system is configured such that the source of electromagnetic radiation emits electromagnetic radiation towards the gemstone from a plurality of different incident directions relative to the external surface geometry of the gemstone.

47. The system of claim 45, wherein the processor is further configured to generate, using the output, a three-dimensional graphical representation of the one or more features associated with the internal structure of the gemstone.

48. The system of claim 45, wherein the processor is further configured to:
generate a first model of a simulated homogenous sample, the simulated homogenous sample comprising a simulated external surface having the external surface geometry of the gemstone, wherein the simulated homogeneous sample has a homogeneous refractive index;
generate a second model of propagation of simulated electromagnetic radiation between the source of incident electromagnetic radiation and the optical detecting means and via a simulated homogeneous sample, wherein interaction between the simulated electromagnetic radiation and the simulated homogeneous sample is accounted for; and
use the first model and the second model to generate the output.

49. A computer program comprising executable code configured to cause the process of the system of claim 45 to execute the steps of:
receiving the first input;
receiving the second input; and
generate the output wherein the output is generated based on for the first input, the second input, and accounting for refraction and reflection effects due to the external surface geometry of the gemstone.
